(19) United States
(12) Patent Application Publication
(10) Pub. No.: US 2020/0028372 A1
(43) Pub. Date: Jan. 23, 2020

(54) CHARGING DEVICE FOR ELECTRONIC CIGARETTE

(71) Applicant: FUUL TECH LLC, Brooklyn, NY (US)

(72) Inventor: Michael Mahana, Brooklyn, NY (US)

(73) Assignee: FUUL TECH LLC, BROOKLYN, NY (US)

(21) Appl. No.: 16/040,125

(22) Filed: Jul. 19, 2018

(51) Int. Cl. H02J 7/00; A61M 11/04; A61M 15/06; H01R 24/60

(52) U.S. Cl. CPC H02J 7/0047; A61M 11/042; H02J 2007/0049; A61M 15/06; H01R 24/60; H02J 7/0052

(57) ABSTRACT

The present disclosure provides a charging device for an electronic cigarette. The charging device comprises a body having a generally rectangular shaped recess which is adapted, at least partially, to accommodate the electronic cigarette inside thereof. The charging device also comprises an electronic circuit located inside the body. The electronic circuit may have one or more pogo pins projecting out therefrom, which are adapted to be in contact with one or more types of charging ports of the electronic cigarette, when the electronic cigarette is accommodated inside the rectangular shaped recess of the body. The charging device further comprises a battery electrically connected to the electronic circuit so as to supply electric power to the electronic cigarette via the one or more pogo pins in contact with the one or more types of charging ports of the electronic cigarette, when the electronic cigarette is accommodated inside the rectangular shaped recess of the body.

### BACKGROUND OF THE INVENTION

### 1. Field of the Invention

The present disclosure generally relates to a charging device, and more particularly, relates to a portable charging device for supplying electric power to charge a power source of an electronic cigarette.

### 2. Description of the Related Art

Electronic cigarettes, also referred to as e-cigarettes, electronic vaping devices or e-vaping devices (EVDs), etc., have become very popular among adults for portable vaping. An electronic cigarette is proposed and used as a substitute for typical cigarettes, such as cigarettes, cigars, and pipe tobacco. The electronic smoking device works by vaporizing a liquid chemical formulation to produce a vapor for inhalation by the user. The produced vapor, when inhaled, has the physical sensation, appearance, and, sometimes, flavor and nicotine content of inhaled tobacco smoke, but without the odor and, arguably, the health risks. Unlike typical cigarettes, the electronic cigarette does not produce harmful substances, such as tar, and does not produce bad odors. As a result, the electronic smoking devices are primarily used as a smoke-less alternative to tobacco smoking, or as an aid for smoking cessation.

A typical electronic cigarette includes a power source, usually a battery therein; and a smoke generating part containing a liquid therein and generating smoke by vaporizing the liquid using electricity supplied from the power source. However, the capacity of the power source, such as the battery, of a conventional electronic cigarette is limited. This is primarily due to the size constraints of the electronic cigarette. That is, in order to make the electronic cigarette easy to carry and to keep its weight to a minimum, the battery used in the electronic cigarette has to be made small. This reduces the number of cells that can be used in the battery, which, in turn, reduces the charge carrying capacity of such battery. The result is that the electronic cigarette can only provide a few, sometimes 2-3, vaping sessions before the battery completely drains out. Thus, the user may have to find a power outlet after few vaping sessions to charge the electronic cigarette via the provided adapter, which may not always be possible, especially when the user is on-the-go. The drained power source could prevent the user from a vaping session when desired, which could be a frustrating experience for the user.

Some references known in the art proposes to solve the said problem in one or more limiting manners. For example, Applicant believes that a related reference corresponds to US Publication Number 20130152954A1 (hereinafter referred to as “the '954 publication”), which provides for an e-cigarette with a battery replacement type that allows a quick recharge and replacement of the battery when it is dead or out of order, by making it a condition that only the battery could be separately drawn out in the inside of the power supply part, in which opening and closing members are formed in an e-cigarette, when a user inhales the smoke that is generated by burning liquid containing nicotine. The use of e-cigarette of the '954 publication may turn out to be a hassle for the user, as the user would need to replace the battery each time it drains out. Furthermore, certain design choices have to be made for such e-cigarette to make the battery removable, which may not always be desirable.

Other documents describing the closest subject matter provide for a number of more or less complicated features that fail to solve the problems described above in an efficient and economical way. None of the documents suggest the novel features of the present disclosure.

### SUMMARY OF THE INVENTION

It is one of the main objectives of the present disclosure to provide a charging device for electronic cigarette which allows for quick and easy charging of the electronic cigarette on-the-go.

It is another objective of the present disclosure to provide a charging device for electronic cigarette which is ergonomic to hold for extended periods and convenient to use.

It is yet another objective of the present disclosure to provide a charging device for electronic cigarette which is portable, inexpensive, and easy to manufacture.

The present disclosure provides a charging device for an electronic cigarette. The charging device comprises a body having a generally rectangular shaped recess formed therein. The rectangular shaped recess is adapted, at least partially, to accommodate the electronic cigarette inside. The charging device also comprises an electronic circuit located inside the body. The electronic circuit has one or more pogo pins projecting out therefrom. The one or more pogo pins are adapted to be in contact with one or more types of charging ports of the electronic cigarette, when the electronic cigarette is accommodated inside the rectangular shaped recess of the body. The charging device further comprises a battery electrically connected to the electronic circuit so as to supply electric power to the electronic cigarette via the one or more pogo pins in contact with the one or more types of charging ports of the electronic cigarette, when the electronic cigarette is accommodated inside the rectangular shaped recess of the body. The electronic circuit is capable of comprising multiple items, including one or more pogo pins, input for a micro-USB port®, a lightning port® and a USB-Type C port®, two rectangular batteries, and a light indicator.

Further objects of the invention will be brought out in the following specification, which detailed description is for the purpose of fully disclosing the invention without placing any limitations thereon.

### BRIEF DESCRIPTION OF THE DRAWINGS

With the above and other related objects in view, the invention consists in the details of construction and combination of parts, as will be more fully understood from the following description when read in conjunction with the accompanying drawings, in which:

FIG. 1 illustrates a diagrammatic planar view of a charging device 100, in accordance with one or more embodiments of the present disclosure;

FIG. 2A illustrates a diagrammatic perspective view of the charging device 100 showing a top side 108 of the body 102 thereof, in accordance with one or more embodiments of the present disclosure;

FIG. 2B illustrates a diagrammatic perspective view of the charging device 100 showing a plate 110 located at a bottom of the body 102 thereof, in accordance with one or more embodiments of the present disclosure;

FIG. 3 illustrates a diagrammatic exploded view of the charging device 100, in accordance with one or more embodiments of the present disclosure; and

FIG. 4 illustrates a diagrammatic sectional view of a body 102 of the charging device 100 taken along a section XX′ of FIG. 2A, in accordance with one or more embodiments of the present disclosure.

### DETAILED DESCRIPTION OF THE EMBODIMENTS OF THE INVENTION

Illustrative embodiments of the present disclosure are described below. The following explanation provides specific details for a thorough understanding of and enabling description for these embodiments. One skilled in the art will understand that the invention may be practiced without such details. In some instances, well-known structures, processes and functions have not been shown or described in detail to avoid unnecessarily obscuring the description of the embodiments.

It shall be noted that unless the context clearly requires otherwise, throughout the description, the words “comprise,” “comprising,” “include,” “including,” and the like, are to be construed in an inclusive sense as opposed to an exclusive or exhaustive sense; that is to say, in the sense of “including, but not limited to.” Words using the singular or plural number also include the plural or singular number, respectively while adhering to the concepts of the present disclosure. Furthermore, references to “one embodiment” and “an embodiment” are not intended to be interpreted as excluding the existence of additional embodiments that also incorporate the recited features.

The same reference numerals throughout the drawings denote elements having the same or similar function. In the following description of the present disclosure, detailed descriptions of known functions and components incorporated herein shall be omitted when it may make the subject matter of the present disclosure unclear or vague. Unless otherwise defined, all terms including technical and scientific terms used herein have the same meaning as commonly understood by one of ordinary skill in the art to which this invention belongs. When terms used herein discord from the commonly understood meaning, the terms shall be interpreted as defined herein.

The embodiments as described herein are subject to many variations. It is understood that various omissions and substitutions of equivalents are contemplated as circumstances may suggest or render expedient, but are intended to cover the application or implementation without departing from the spirit or the scope of the present disclosure. Further, it is to be understood that the phraseology and terminology employed herein are for the purpose of the description and should not be regarded as limiting. Any heading utilized within this description is for convenience only and has no legal or limiting effect.

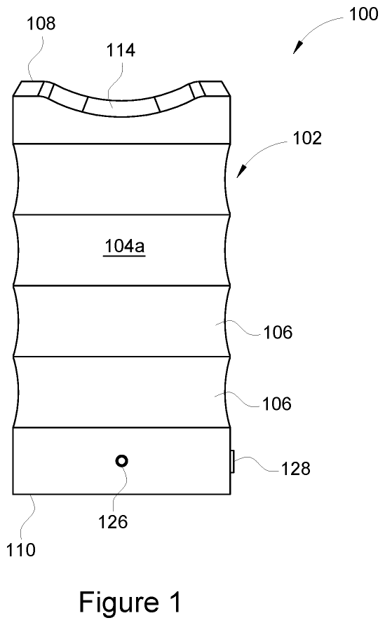

Referring to the drawings, FIG. 1 illustrates a diagrammatic view of a charging device (referred by the numeral 100), in accordance with one or more embodiments of the present disclosure. The charging device 100 has been designed to be used for charging of an electronic cigarette. In particular, the charging device 100 has been designed to be used with a popular type of electronic cigarette sold in the market under the brand name of “Juul” or some variation thereof. Although the present disclosure has been described in terms of the charging device 100 being implemented for charging of the said brand of the electronic cigarette, it may be understood that the presently disclosed charging device 100 can be used with other types of electronic cigarettes with or without making some design modifications. The charging device 100 of the present disclosure can conveniently charge a power source of the electronic cigarette without the need of disassembling the electronic cigarette, thereby making the charging of the electronic cigarette a hassle-free experience for the user.

As illustrated in FIG. 1, the charging device 100 includes a body 102. In the illustrated embodiments, the body 102 may have a generally rectangular planar shape; however, in other examples, the body 102 may have any other suitable shape, including a circular shape, without any limitations. The body 102 may have four sides, with only one side 104a shown in FIG. 1. The body 102 may be made of plastic material or the like which provides good strength while having less weight. It may be contemplated that the body 102 may be manufactured by any suitable process, such as, but not limited to, molding. In one or more examples, the body 102 may be coated with a rubber material at exterior thereof. Such rubber material may provide grip to the user for holding the charging device 100 in his/her hand. In other examples, the body 102 in itself may be made of silicone material or the like for providing better grip for the user. Further, in one or more examples, the body 102 may include finger grooves 106 formed on one or more sides, such as the side 104a, at the exterior thereof. Such finger grooves 106 provide spaces for the user to put his/her fingers while holding the charging device 100, thus making the charging device 100 ergonomic to hold during long periods of use.

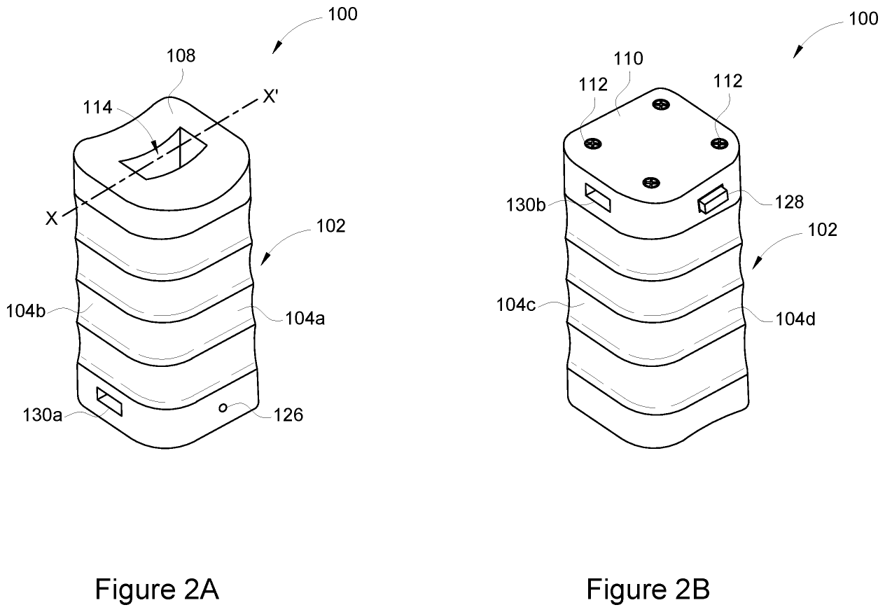

FIGS. 2A-2B illustrate two different perspective views of the charging device 100. In an embodiment, as illustrated in FIG. 2A, a top side 108 of the body 102 may have a curved profile. Further, as illustrated in FIG. 2B, a bottom of the body 102 is provided with a plate 110 which is coupled to the body 102 by means of screws 112. The plate 110 may be a flat plate in order to allow for the charging device 100 to stand while laid on a flat surface, such as a table top or the like. Referring to FIG. 2A, as illustrated, the body 102 may have a generally rectangular shaped recess 114 formed in the top side 108. The rectangular shaped recess 114 is adapted, at least partially, to accommodate an electronic cigarette, such as the said electronic cigarette of the brand name “Juul,” inside thereof. It may be understood that the rectangular shaped recess 114 is dimensioned so as to allow for the electronic cigarette to easily slide inside the body 102 and further adapted to, at least partially, accommodate the electronic cigarette inside thereof.

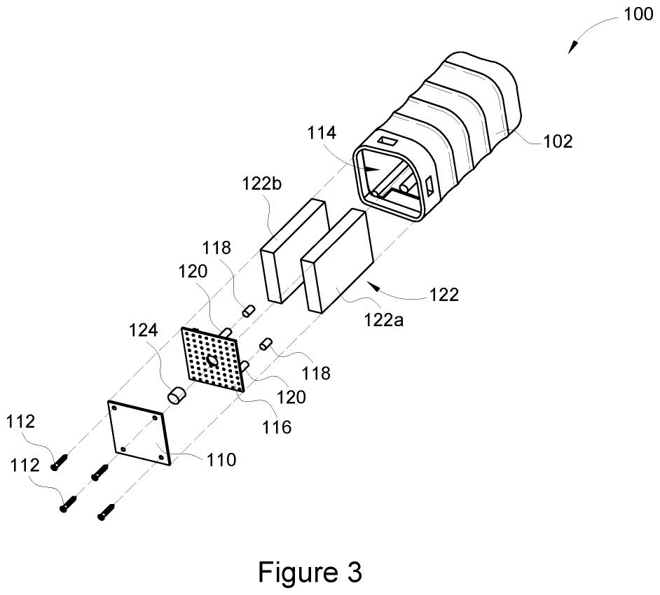

FIG. 3 illustrates an exploded view of the charging device 100 showing the various components located inside the body 102. As illustrated, the charging device 100 includes an electronic circuit 116. The electronic circuit 116 is adapted to be positioned inside the body 102 right above the plate 110. In the illustrated embodiments, the electronic circuit 116 may have one or more pogo pins 118 projecting out therefrom; however, in other examples, the electronic circuit 116 may use other forms of electronic contacts sufficient to supply electric power. The one or more pogo pins 118 are adapted to be in contact with one or more types of charging ports of the electronic cigarette, when the electronic cigarette is accommodated inside the rectangular shaped recess 114 of the body 102. In one example, the one or more pogo pins 118 may be biased by means of springs 120 associated with the electronic circuit 116. In one example, the

Further, as illustrated in FIG. 3, the charging device 100 includes a battery 122 electrically connected to the electronic circuit 116. The battery 122 is configured to supply electric power to the electronic cigarette via the one or more pogo pins 118 in contact with the one or more types of charging ports of the electronic cigarette, when the electronic cigarette is accommodated inside the rectangular shaped recess 114 of the body 102. In one or more examples, the battery 122 may include two rectangular batteries 122a and 122b arranged on two of opposite sides inside the body 102 to define the rectangular shaped recess 114 therein. In other examples not illustrated, the battery 122 may include one or more batteries, and batteries of various shapes. In some examples, the charging device 100 may further include a magnet 124 positioned inside the body 102 so as to keep the electronic cigarette in place inside the rectangular shaped recess 114 thereof. As illustrated, the magnet 124 may be located at the bottom, associated with the electronic circuit 116, proximal to the plate 110 inside the body 102. It may be understood that the magnet 124 may grab the electronic cigarette, which usually has a metallic body, in order to prevent the electronic cigarette from slipping and falling out of the rectangular shaped recess 114 without user intervention. This may help to keep the electronic cigarette held and in place with respect to the charging device 100 while being charged, for example when in user's pocket or when the charging device 100 is held upside down.

Again, referring to FIG. 2A, the charging device 100 includes a light indicator 126 provided on the side 104a at the exterior of the body 102. The light indicator 126 is electrically coupled with the electronic circuit 116. The light indicator 126 is configured to indicate one or more of charging and discharging of the battery 122 inside the body 102 of the charging device 100, for example by illuminating in different colors. Such LED indicators are well known in the art and have not been described herein. Referring to FIG. 2B, the charging device 100 also includes a power button 128 provided on the side 104d at the exterior of the body 102. The power button 128 is electrically coupled with the electronic circuit 116. The power button 128 is configured to activate the electronic circuit 116 to supply electric power from the battery 122 to the electronic cigarette when in a depressed position and to stop the supply of electric power from the battery 122 to the electronic cigarette otherwise.

Now referring to FIGS. 2A-2B in combination, the charging device 100 includes at least one type of charging port 130 located at the exterior of the body 102. The at least one type of charging port 130 is electrically connected to the electronic circuit 116 located inside the body 102. The at least one type of charging port 130 allows for electrical connection between a main power supply, such as a power socket in the home, and the electronic circuit 116 for receiving electric power for charging of the battery 122 located inside the body 102. The at least one type of charging port 130 may include two or more types of a micro-USB port®, a lightning port® and a USB-Type C port®, as known in the art. In one embodiment, the charging device 100 includes two types of charging ports 130a and 130b located on two sides 104b and 104c, respectively, at the exterior of the body 102. In one exemplary configuration of the charging device 100, the two charging ports 130a and 130b may be a micro-USB port® and a lightning port®, respectively. In some examples, the charging device 100 may further be provided with solar panels (not shown) at the exterior of the body 102 to generate electric power for charging of the battery 122 therein.

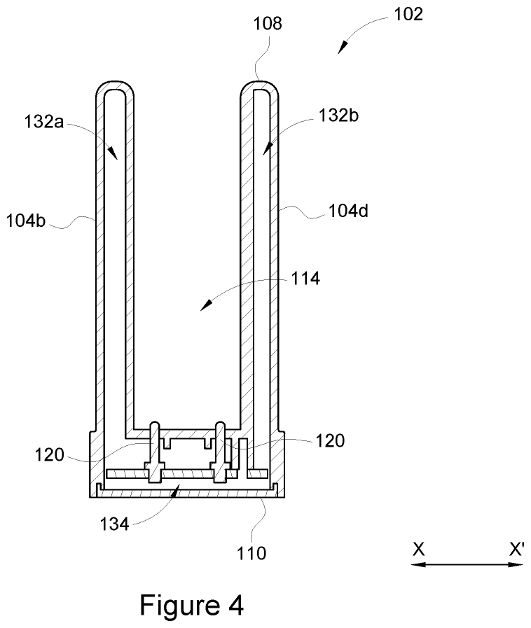

FIG. 4 illustrates a sectional view of the body 102 of the charging device 100 taken along a section XX′ of FIG. 2A. As illustrated, the body 102 provides two sections 132a and 132b formed along the length at the inside of the two sides 104b and 104d respectively. Each of the two sections 132a and 132b may be adapted to receive each one of the two rectangular batteries 122a and 122b therein. As may be seen, the rectangular shaped recess 114 is defined between the sections 132a and 132b inside the body 102. Further, the body 102 provides a section 134 adapted to position the electronic circuit 116 therein. As may be seen, the springs 120 may be provided above the section 134 to be associated with the electronic circuit 116 and the one or more pogo pins 118 when arranged inside the body 102.

The charging device 100 is a portable electronic cigarette charger which allows one to slide the electronic cigarette inside the body 102 for charging purposes. The charging device 100 is portable enough to be easily carried by the user for regular use and travel due to the compact arrangement of the various components inside the body 102 thereof, thus allowing for on-the-go charging of the electronic cigarette. The charging device 100 may provide up to 3-6 full charges of power source for the electronic cigarette from its completely charged battery 122, without any wires or power outlet. The ergonomic design of the body 102 with finger grooves 106 ensures that the charging device 100 can comfortably fit in user's hand and the rubber texture of the body 102 makes the charging device 100 feel nice and comfortable in user's hand. This ensures that the user could hold the charging device 100 for extended periods of time while using the electronic cigarette.

Since the electronic cigarette is placed inside of the body 102 for charging purposes, the charging device 100 ensures that the electronic cigarette does not fall off. Further, the magnet 124 grabs the electronic cigarette and attaches the electronic cigarette with the one or more pogo pins 118 to start charging the electronic cigarette. The charging device 100 provides the power button 128 which may be pressed into depressed position by the user to turn on the charging of the electronic cigarette, and further pressed to stop the charging of the electronic cigarette.

The power button 128 allows the user to use the electronic cigarette while the electronic cigarette is inside the body 102 by pressing the power button 128 to stop the charging of the electronic cigarette.

The charging device 100 provides the user with an option to charge its battery 122 via a choice of one or more types of charging ports, such as a micro-USB port and a lightning port®. One embodiment of the charging device 100 may have at least one type of charging port; a second embodiment of the charging device 100 may have two types of charging ports 130a and 130b; and a third embodiment of the charging device 100 may have multiple types of charging ports. So, the user can choose to charge the battery 122 from any power cord available therewith.

While the preferred embodiment of the present disclosure has been illustrated and described, as noted above, many changes can be made without departing from the spirit and scope of the present disclosure. Accordingly, the scope of the present disclosure is not limited by the disclosure of the preferred embodiment. Instead, the invention should be determined entirely by reference to the claims that follow.

What is claimed is:

1. A charging device for an electronic cigarette, the charging device comprising:
a body having a generally rectangular shaped recess formed therein, the rectangular shaped recess adapted, at least partially, to accommodate the electronic cigarette inside thereof;
an electronic circuit located inside the body, the electronic circuit having one or more pogo pins projecting out therefrom, the one or more pogo pins adapted to be in contact with one or more types of charging ports of the electronic cigarette, when the electronic cigarette is accommodated inside the rectangular shaped recess of the body; and
a battery electrically connected to the electronic circuit so as to supply electric power to the electronic cigarette via the one or more pogo pins in contact with the one or more types of charging ports of the electronic cigarette, when the electronic cigarette is accommodated inside the rectangular shaped recess of the body.

2. The charging device of claim 1 further comprising at least one type of charging port located at exterior of the body and electrically connected to the electronic circuit located inside the body, the at least one type of charging port allows for electrical connection between a main power supply and the electronic circuit for receiving electric power for charging of the battery inside the body.

3. The charging device of claim 2, wherein the at least one type of charging port comprises two types of charging ports located on two sides at the exterior of the body.

4. The charging device of claim 2, wherein the at least one type of charging port comprises two or more types of a micro-USB port®, a lightning port® and a USB-Type C port®.

5. The charging device of claim 1, wherein the battery comprises two rectangular batteries arranged on two of opposite sides inside the body to define the rectangular shaped recess therein.

6. The charging device of claim 1 further comprising a magnet positioned inside the body so as to keep the electronic cigarette in place inside the rectangular shaped recess thereof.

7. The charging device of claim 1, wherein the body comprises finger grooves formed on one or more sides at exterior thereof.

8. The charging device of claim 1, wherein the body is coated with a rubber material at exterior thereof.

9. The charging device of claim 1 further comprising a power button provided on one of sides at exterior of the body and coupled with the electronic circuit, wherein the power button is configured to activate the electronic circuit to supply electric power from the battery to the electronic cigarette when in depressed position.

10. The charging device of claim 1 further comprising a power button provided on one of sides at exterior of the body and coupled with the electronic circuit, wherein the power button is configured to allow the user to use the electronic cigarette while the electronic cigarette is inside the body by pressing the power button to stop the charging of the electronic cigarette.

11. The charging device of claim 1 further comprises a light indicator provided on one of sides at exterior of the body and coupled with the electronic circuit, wherein the light indicator is configured to indicate one or more of charging and discharging of the battery inside the body.
